(19) United States
(12) Patent Application Publication
(10) Pub. No.: US 2004/0052850 A1
(43) Pub. Date: Mar. 18, 2004

(54) Proteinaceous hemostatic tissue sealant

(76) Inventor: Kemal Schankereli, Stillwater, MN (US)

Correspondence Address: BOZICEVIC, FIELD & FRANCIS LLP, 200 MIDDLEFIELD RD, SUITE 200, MENLO PARK, CA, 94025, US

(21) Appl. No.: 10/243,482

(22) Filed: Sep. 13, 2002

(51) Int. Cl. A61K 31/74; A61K 9/14

(52) U.S. Cl. 424/486

(57) ABSTRACT

A sealant useful for the control of fluid leakage in bloody and non-bloody surgical fields.

### BACKGROUND OF THE INVENTION

I. Field of the Invention

This invention relates generally to tissue sealants for use in surgical procedures, and more particularly to a sealant formulation and process that results in a product useful in both wet and dry fields.

II. Discussion of the Prior Art

Control of fluid and gaseous leakage at a surgical site is a factor critical to the successful outcome to any invasive procedure. Historically, numerous methods have been deployed to assist the surgeon in this task including the use of hemostatic sponges, the use of hemostats agents such as fibrin/thrombin, the placement of drains and the use of sutures. Depending upon the conditions present at the time of surgery one or all of these devices would be used by the clinician to optimize the surgical outcome.

Recently, a number of sealants have become available to control fluid leakage at a surgical site. However, each of these has serious limitations with regards to the field in which they can be used as well as their biocompatibility and their physical properties. Side effects, such as inflammation, acute fibrous formation at the wound site, toxicity, inability to be used in a bloody field, poor physical properties of the sealant, and poor adhesion to the surgical site, may have a serious impact on the patient and resultantly may play a significant role in the long term efficacy of the repair. Further, useful sealants have properties that render them more effective for surgical application. Characteristics, such as the ability to be localized to a specific location, adequately long or short polymerization times, and adequate in vivo resorption characteristics, are vital to a successful completion of the sealing procedure.

Various synthetic sealants and adhesives have been offered to solve wound sealant problems. One group of sealants is based on various 2-cyanoacrylate formulations (U.S. Pat. No. 3,559,652) and 2 alkoxyalkylcyanoacrylate (U.S. Pat. No. 6,299,631). These glues often contain a component of the cyanoacrylate group polymerized by polyethylene glycol.

The disadvantages of cyanoacrylate adhesives typically include toxicity, exothermic reactions when polymerized, poor bioresponses, and poorly controllable biodegradation responses. Degradation of this material generally leads to the creation of short chain polymers (monomers, dimers, trimers and oligomers) of cyanoacrylate that can promote strong inflammatory responses leading to reduced biocompatibility.

Still further, cyanoacrylates do not adhere well in wet or moist environments and do not, in and of themselves, exhibit strong platelet aggregation activity. Platelet aggregation is necessary for the formation of blood clots since it is the aggregation activity that ultimately causes the degranulation activities of platelets necessary to initiate the polymerization of blood fibrin in the presence of thrombin. Resultantly, cyanoacrylates cannot be used in wet or bloody surgical fields since they do not exhibit attachment to tissues in these circumstances and thus will not stop fluid flow. Cyanoacrylate adhesives work best in situations where the fluid flow is minimal and there is a slightly moist, but not wet, environment. One example of such an environment would be the application of the adhesive at a wound requiring sealing at an air interface.

Still another group of sealants are produced from components found to be a part of the naturally occurring sealing/clotting mechanism in mammalian systems. These sealants, which are based on a combination of fibrin, thrombin and calcium, are advantageous for use in fields where bleeding is apparent since they incorporate the use of autologous clotting proteins to enhance their sealing potential. In this clotting reaction, the blood protein, fibrinogen, in the presence of the crosslinker, prothrombin, is cleaved to form the clot. The reaction requires the presence of a calcium catalyst. Thus, thrombin acts as a natural crosslinker for the reaction. While a clot created using a fibrin sealant can be used more effectively in a bloody field than, for instance, a cyanoacrylate sealant, it does not have high tensile strength and it's adherence to the underlying surface is minimal. Thus, its applicability at sites where high fluid pressures or high tear strength are required is limited. As indicated, fibrin sealants, though effective for sealing in bloody fields, exhibit low tear strengths, and poor adhesion to the underlying surface. In addition, these seals exhibit poor elasticity. Thus, their utility in applications where frequent expansion and contraction might be encountered, for example pulmonary applications, would be compromised.

Still another group of sealants is comprised of proteinaceous materials that can be crosslinked to create biological polymers. U.S. Pat. No. 5,219,895 indicates one such system in which the protein, collagen, is polymerized using a sulfonating agent. The material created exhibits sealant properties in that it will stick to a surface in the absence of blood. The presence of blood in this field, in contrast to what is noted for fibrin, is deleterious to the adhesive properties of the sealant. Non-fibrin, proteinaceous sealants will adhere to non-bloody surfaces with greater tenacity than fibrin sealants, but do poorly in bloody applications. Numerous crosslinking agents are useful for crosslinking proteins. One such agent is glutaraldehyde. The creation of Schiff's bases in which the carbonyl terminal of glutaraldehyde attaches to a primary amine, such as might be exhibited on the amino acids, lysine, arginine or histidine, creates a proteinaceous polymer which has applicability as a sealant. However, sealants created using monomeric glutaraldehyde may ultimately exhibit poor bioresponses, partially due to the reversal of the crosslinking process and the resultant presence of glutaraldehyde in the surrounding microenvironment. For maximum biocompatibility to be achieved, the Schiff's bases created must be reduced.

Thus, we have seen that when addressing the need for a sealant, at the time of surgery a clinician must consider the state of the surgical field—bloody or not, the need for flexibility in the sealant, the need for strength of the adhesion to the underlying surface, resorption characteristics required, and a host of other considerations.

To date, no single material has addressed itself to all of these issues.

### SUMMARY OF THE INVENTION

This invention provides a method of producing a proteinaceous sealant that is useful for the bonding and sealing of tissues in both wet (bloody) and dry (non-bloody) fields. It discloses a method of controlling the durometer of the sealant, and further, the tenacity of the sealants' adhesions to the underlying tissue. It is further an intention of this invention to disclose a method of producing crosslinking agents with reduced toxicity, while at the same time, improving the ability of the sealant to crosslink the proteinaceous substrates constituting the substrate of the sealant.

The present invention relies on the use of proteins, carbohydrates and tacking materials to form the substrate of the sealant. Proteins found to be useful in this application include albumin, and soluble and insoluble forms of collagen and elastin. These proteins may be from any mammalian source, but sources specifically advantageous to this invention because of their wide availability include bovine and human. The protein concentration is normally between about 1 and 50% (w/w). Carbohydrates found to be useful in the formation of sealants, and specifically useful for the formation of clots in a bloody field, include chitin and chitosan and its' derivates. Carbohydrate concentration is preferably between 1 and 5% (w/w).

Tacking agents improve the ability of the proteins to be attached to the surface of the surgical site. One particularly useful tacking agent is polyethyleneimine (PEI). The propensity of primary, secondary and tertiary amine terminals in this material allows strong ionic interactions with any surface with which the material comes into contact. In addition, the presence of amine terminals in the presence of a carbonyl functionality such as might be displayed in an aldehyde, allows for covalent crosslinking to a surface, as well. Other adhesion modifiers include, for example, gelatin and carboxymethylcellulose.

While other materials such as monocarbonyl compounds can be used, the preferred crosslinking agents are dicarbonyl compounds, of these most preferably are dialdehydes, such as glutaraldehyde.

The present invention further includes a method for producing an aldehyde derived crosslinker with improved chemical stability by careful heating of concentrations of the material to form cyclic compounds not requiring further reduction, once the Schiff's base response has been completed. In particular, the heat treatment of the crosslinker is associated with improved bioresponses of the resultant sealant.

Sealant material of the invention also contemplates the optional addition of fatty acid materials as plasticizers. These include materials such as polyethylene glycol, glycerin, oleic acid or palmitic acid.

The combination of proteins, lipids, carbohydrates, tacking agent, and crosslinker described in this application have been found to have exceptionally strong adherence to the underlying surfaces when used in dry fields. Alternately, when used in wet applications, the material has been found to be an aggressive coagulator of blood.

The present invention is advantageous in the handling of fluid leaks in a broader range of applications without penalty of consideration of the site conditions.

The current invention also includes useful kits including substrate and crosslinker based on the preferred composition of the chemical components and the intended surgical application of the material.

One embodiment of the invention is a method of preparing the material comprised of mixing substrate materials, including tacking agents, proteins, and carbohydrates, with prepared crosslinker. The substrate and crosslinkers are packaged in separate syringes. At the time of use, the components are mixed and applied to the site to be sealed.

### DESCRIPTION OF THE PREFERRED EMBODIMENT

Composition of the Sealant

The sealant of this invention is comprised of proteins, carbohydrate, fatty acids and synthetic components. The proteins of this invention may be derived from either synthetic or natural sources, but proteins particularly derived from human and bovine sources have been found to be advantageous because they are abundantly available and effective in sealant applications.

The preferred proteins of this invention include albumin, and collagen in concentrations ranging from 1-50% (w/w). The specific selection of concentration is dependent on the desired application. Considerations, such as tenacity, hardness, elasticity, resorption characteristics and platelet aggregation effects, will be determinant with regards to the ultimate concentrations for each of the proteins.

The primary protein of the sealant composition of this invention is albumin in concentrations ranging from 10-50% (w/w), but preferably in the ranges of 30-40% (w/w). The concentration of collagen may range from 1-20% (w/w) but is preferably in the range of 2-4% (w/w). The albumin may be purchased in powdered form and the solubilized into an aqueous suspension, or alternately, may be purchased in aqueous form.

Purified albumin may derived from any one of a number of different sources including, bovine, ovine, equine, human, or avian in accordance to well known methods (ref.: Cohn et. Al, J. Amer. Chem. Soc. 69:1753) or may be purchased in purified form from a supplier, such as Aldrich Chemical (St. Louis, Mo.), in lyophilized or aqueous form. In the preferred embodiment of this invention pure aqueous/albumin concentrations of 30-40% (w/w) may be purchased from a suitable supplier.

In accordance to the invention, the albumin may be derivatized to act as a carrier for drugs, such as heparin sulfate, growth factors, antibiotics, or may be modified in an effort to moderate viscosity, or hydrophilicity. Derivitization using acylating agents, such as, but not limited to, succinic anhydride, and lauryl chlorides, are useful for the creation of binding sites for the addition of useful molecules.

According to the invention, collagen may be included in the proteinaceous complex of the sealant. Collagen, in either soluble or insoluble form, may be used.

The concentration of the collagen can be between 1-10% (w/w), but is preferably is between 1-4% (w/w). In accordance with the invention, the collagen may be in dry or aqueous forms when mixed with the albumin.

Collagen may be derivatized to increase it utility. Acylating agents, such anhydrides or acid chlorides, have been found to create useful sites for binding of molecules such as growth factors, and antibiotics.

Preparation of the Crosslinker

According to the invention, a crosslinker is used to polymerize and stabilize the proteinaceous substrate of the invention. Crosslinking refers specifically to the creation of a bond between adjacent functional groups for purposes of rendering the molecule less susceptible to chemical degradation. Crosslinking resultantly affects the resorption characteristics of the sealant substrate as well as the biological responses induced by the presence of the sealant. Numerous crosslinking agents have been identified. Examples of these are photo-oxidative molecules, carbodimides, and aldehydes.

Whereas, any aldehyde crosslinker may be used to crosslink the substrate,

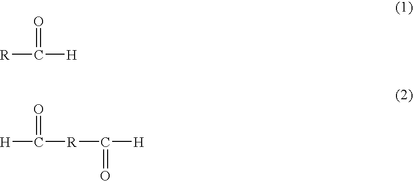

where R(CH₂)ₙ

n≧1

In general aldehydes react with amines to form Schiff's bases in accordance to the generalized formula:

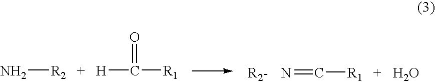

1°,2° 3° Amine (+) Aldehyde Schiff's Base

R₁ May be H or Alkyl

Lysine Arginine R₂ May be: H, Alkyl or Aryl Groups

Aldehydes are reactive over a wide pH range (1-9.5). However, in general, optimal crosslinking occurs in the pH range of 5-8 with pH's of 6-7 being particularly useful. At either low or high pH values, the reaction of an aldehyde with an amine is reduced. Sensitivity with regards to pH, thus, is one variable that can be used to control the reaction rates and density of crosslinking. Other variables include time, temperature, and concentration of the crosslinking solution. Density of crosslinking can be measured using a number of different assay techniques or by the physical measurement of tensile strength of the crosslinked polymer (sealant).

Still another variable affecting the density of crosslinking and reaction rates relates to the presence of the number of amine terminals available for reaction. Proteins are comprised of amino acids. Certain amino acids contain amine terminals in abundance. These include the amino acids lysine, arginine and histidine. Exposure of an aldehyde with an available primary, secondary or tertiary amine, such as might be contained on the amino acids, lysine, arginine and histidine, will result in the formation of a Schiff's base. The stability of the formed bond is partially dependent upon the pH conditions at the time of reaction and can also be modified, i.e., enhanced, by the use of a reducing agent when creating the bond. One example of a reducing agent that has been found to be useful for this purpose includes, sodiumborohydride and it's derivatives. Treatment of a Schiff's base with sodiumborohydride stabilizes any reversible reactive group and reduces any residual aldehyde in accordance to the generalized formula:

NaBH₄+4CH₂═O+H₂O→4CH₃OH+NAB(OH)₄ (4)

Though sodiumborohydride is a useful agent for the stabilization of any reversible bonds, it has not been widely accepted for use in part due to difficulties in handling and secondarily, because of suspect biocompatibility affects.

One aldehyde useful for the creation of Schiff's bases is the water soluble dialdehyde commonly referred to glutaraldehyde. Glutaraldehyde strongly binds any primary amine terminal. However, it may dissociate over time if not properly reduced. The use of borohydride subsequent to the creation of the Schiff's base will create a more stable bond. However, borohydride reduction is difficult to conduct, is time consuming and costly and as indicated earlier, may negatively affect biocompatiblity.

According to the invention, treatment of the glutaraldehyde prior to use for crosslinking can render the molecule more stable post crosslinking, eliminating the need for reduction.

Proper crosslinking, in accordance to the invention, requires that the dialdehyde, be in such a form that it's structure is predictable. In the preparation of a dialdehyde, such as glutaraldehyde and in anticipation of it's use for crosslinking, a concentration of glutaraldehyde is mixed with water at a given pH. Whereas, the resultant aqueous mixture of glutaraldehyde is generally believed to be largely monomeric glutaraldehyde, some dimmers, trimmers, and oligomers of glutaraldehyde are formed as well. Further, subsequent to the initial formation of the largely monomeric glutaraldehyde solution, conditions including pH, time, temperature, and concentrations will affect the relative subsequent to the initial formation of the largely monomeric glutaraldehyde solution, conditions including pH, time, temperature, and concentrations will affect the relative concentrations of each of these structures in the solution. Initially, the monomeric form of glutaraldehyde is favored in solution. However, over time; binding of the monomers in solution will form short chain polymers with different crosslinking characteristics than those noted in the monomeric form of glutaraldehyde. Thus, it is clear that the chemical configuration of glutaraldehyde may be modified in accordance with the preparation process and that glutarldehyde changes continually in solution. The monomeric form, often desirable for crosslinking applications, is short lived, quickly reverting to a polymerized form less useful for crosslinking.

According to the invention, a stabilized dialdehyde moiety useful for crosslinking can be created by heating a solution of glutaraldehyde for a period of time. In the preferred embodiments of the invention, a 1-20% aqueous solution of glutaraldehyde pH 7.0 is prepared in accordance to standard methodology. Preferably, the solution is in the range of 7-12%. An aliquot of solution is placed into an air-tight flask under a nitrogen head and heated in an oven at a temperature of 35-60° C., and preferably 45-55° C. for a period of 1-14 days. Glutaraldehyde treated in this manner will form a pyridinium complex indicated by the generalized chemical formula:

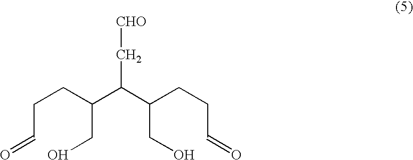

Reaction of the pyridinium complex with the reactive anabilysine, arginine or histidine will result in the structue indicated in formula 6.

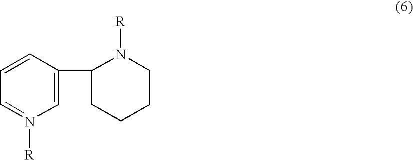

Structure 6 indicates the ring structures formed upon completion of the pyridinium crosslinks—anabilysine. A more complete understanding of the formation of the stable crosslinks created by anabilysine may be imparted by reviewing the formation of the intermediates.

Equations (7), (8) and (9) indicate the formation of the pyridinium complexes and ultimately the creation of a stable crosslink without need for the use of any additional reducing agents.

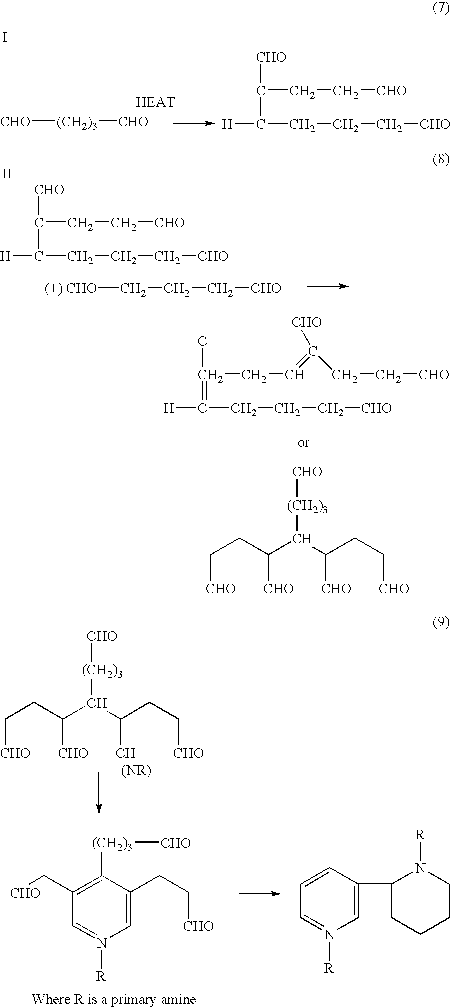

The heating time is preferably 72-120 hours. Following heating, the solution is cooled to room temperature and is used for crosslinking of proteins. Schiff's bases created in this manner do not require the use of any additional reducing agents to stabilize their bonds, since the heat-treated dialdehyde is electrovalently in a stable form. Crosslinks created using heat-treated dialdehydes are covalently bonded structures and are resultantly less susceptible to reversal. Thus proteins crosslinked using heat treated glutaraldehyde are more stable and do not exhibit the intense inflammatory responses noted as a result of the reversal of crosslinks when using non-heat treated dialdehydes.

It should be noted that, while the detailed embodiment described is limited to glutaraldehyde, it is contemplated that other, similar aldehydes such as succinaldehyde could successfully be employed as crosslinkers in the sealant preparation of the invention.

Buffering of the sealant solution is important to optimize the bonding strength of the sealant to the attaching surface while simultaneously optimizing the conditions necessary for internal crosslinking to occur. For example, optimum crosslinking for proteins using glutaraldehyde crosslinkers occurs at pH's 6-8. Buffers capable of maintaining this range are useful in this invention, as long as they do not interfere with the carbonyl terminal of the crosslinker or modify the amine terminus of the amino acids. For example, phosphate buffers have a pKa value in the range of pH 7.0 and would not be expected to interfere with the crosslinking process since they do not contain carboxylic or amine functionalities. Conversely, for example, TRIS buffers would not be recommended since they do contain interfering molecules. Phosphate buffer up to 1 M in strength is suitable for this invention. Since phosphate buffers do not contain primary, secondary or tertiary amines, they would not be expected to interfere in the crosslinking process.

In the preferred embodiment of this invention, phosphate buffer, 0.2M in strength, is used to modify the sealant solution to further enhance intra-molecular crosslinking of the sealant and make the sealant more compatible with biological tissues.

While phosphate buffering of the solutions is ideal for the stability of the protein substrate in applications where increased adhesion is required, an acidic buffer may be used without penalty. Citrate buffers 0.1-1M and in the pH ranges of 4.5-6.5 have been found to be useful for this invention. The concentration of the buffer should be determined through experiment by analysis of the adhesion characteristics of the prepared sealant to a characterized tissue surface.

Plasticizing Agents

In accordance with the invention, a plasticizing agent may be used to with the sealant. The plasticizing agent is used to increase the wetting of a surface, or alternately, to increase the elastic modulus of the material, or further still, to aid in the mixing and application of the material. Numerous plasticizing agents exist, including oleic acid, palmitic acid, dioctylphtalate, phospholipids, and phosphatidic acid. Because plasticizers are typically water insoluble organic substances and are not readily miscible with water, it is sometimes advantageous to modify their miscibility with water, by pre-mixing the appropriate plasticizer with an alcohol to reduce the surface tension associated with the solution. To this end, any alcohol may be used. In one embodiment of this invention, oleic acid is mixed with ethanol to form a 50% (w/w) solution and this solution, then is used to plasticize the proteinaceous substrate of the sealant during the formulation process. Whereas the type and concentration of the plasticizing agent is dependent upon the application, the preferred final concentration of the plasticizing agent is 0.01 to 10% (w/w) and is preferably in the range of 2-4% (w/w).

Adhesives/Tacking Agents

Tacking agents may be used to modify the adhesiveness of the sealant to the biological surface. The tacking agent may be added to the biological surface as a primer prior to the application of the sealant or, alternately, may be incorporated directly into the substrate of the product. Whereas numerous tacking agents may be used, one of particular applicability is polyethyleneimine (PEI). PEI is a long chain branched, alkyl polymer containing primary, secondary and tertiary amines. The presence of these highly ionic groups create significant attachment through ionic interactions with the underlying surface. In addition, the presence of PEI in the sealant substrate significantly enhances the presence of amine terminals suitable to create crosslinks with the prepared dialdehyde.

As indicated, PEI has a significant ionic charge associated with it. Given the presence of amine terminals, the net charge of this molecule is positive. In addition to the indicated intended effect of improving adhesion of a molecule to a surface, a positively charged molecule will have a second important application in a sealant product. Positively charged molecules have long been indicated to be coagulators of blood because they initiate the serine protease coagulation cascade. Hence, the addition of non-crosslinked or lightly crosslinked PEI in the sealant solution will enhance not only the adhesion of the sealant to the underlying surface but has a procoagulant effect.

In the preferred embodiment of the invention, tacking agents are used to modify adhesion to the biological substrate while simultaneously creating a procoagulant. Tacking agents, when used, are apparent in the concentrations of 0.1-10% (w/w). Preferably the tacking agent is evident in 0.5-4% (w/w) concentrations.

Carbohydrate Procoagulant

According to the invention, a sealant should function in a surgical field in which blood is present. Chitosan and derivates of chitosan are potent coagulators of blood and, therefore, are beneficial in formulating sealant materials capable of sealing vascular injuries. While virtually all chitin materials have been demonstrated to have some procoagulant activity, in accordance to the invention, the use of acetylated chitin is preferable as an additive for the formulation of sealant intended for blood control. Acetylation of the molecule can be achieved in a number of different ways, but one common method is the treatment of chitosan/acetic acid mixtures with acid anhydrides, such as succinic. This reaction is readily carried out at room temperature. In accordance to the invention, gels created in this manner combined with proteinaceous substrates and crosslinked in situ are beneficial for the creation of a sealant.

Chitosan reacts with aldehydes to yield an aldimine. The corresponding product can be further hydrogenated to control the hydrolysis of the product. Fully hydrogenated products are less susceptible to hydrolysis than those that have not been hydrogenated. In accordance with the invention, the hydrogenation of the Schiff base can be controlled, either by using heat treated Glutaraldehyde solution prepared in accordance to the methodology taught in this application, thus yielding a stable implant product, or alternately, by using fresh, non-heat treated glutaraldehyde for creating the Schiff base product thus creating a product that slowly re-sorbs in vivo.

While it is understood that the sealant may consist of any form of chitosan, an acetylated molecule is preferred because of the procoagulant activities of this moiety.

In accordance with the teachings of this invention chitosan maybe present in the concentrations of 0-20% (w/w), but is preferably in the range of 2-5% (w/w).

### EXAMPLES OF PREFERRED COMPOSITION

In accordance to the invention, a useful sealant substrate formulation consists of base proteins, preferably albumin and collagen, an adhesion modifier, preferably polyethyleneimine, a plasticizer preferably oleic acid, a procoagulating carbohydrate, preferably chitosan. The substrate of the sealant is crosslinked with an aldehyde solution, which may be any aldehyde but is preferably glutaraldehyde, which has been heat treated to form anabolysine.

According to the invention albumin concentrations can be 10 to 50, but may preferably be between 30 to 40% (w/w). Collagen concentrations can be 1 to 20% (w/w), but preferably is between 2 and 4% (w/w). PEI concentrations can be 0.1 to 10% (w/w), but is preferably between 0.5 and 4% (w/w). Plasticiczer (oleic acid) concentrations are preferably between 0.1 to 10% (w/w), but is preferably between 2 and 4% (w/w). Chitosan concentrations can be 0.01 to 20% (w/w), but are preferably between 2 and 5% (w/w).

The chitosan of this invention may be prepared to be acetylated and gelatinous by pretreating with an acid and alcohol and then exposing the material to an anhydride, such as succinic anhydride.

While the crosslinker in of this formulation may be any aldehyde, heat processed glutaraldehyde has been found to be particularly effective. A 1.5% (w/w) concentration of glutaraldehyde/water is placed into a flask under nitrogen. The mixture is heated in a convection oven at 50° C. for a period of 24-120 hours, but preferably in the range of 70-80 hours. Thereafter, the mixture is allowed to cool to room temperature and is used as is in the subsequent crosslinking of tissue.

Ultra violet spectrographic evaluation of the glutaraldehyde solution, heat treated in accordance to the method of this invention, indicates a reduction of the monomeric form of the molecule and the concurrent increase in the cyclic form of the molecule. Starting with a 1.5% (w/w) solution of monomeric glutaraldehyde, treated in accordance to the method of the invention, the monomeric concentration was reduced to approximately 0.8% (w/w) following 72 hours of heat treatment.

Experiment No. One

Heat-treated glutaraldehyde was evaluated to determine crosslinking efficiency. Glutaraldehyde solution (5% w/w) was used to crosslink a solution containing 35% (w/w) albumin. The albumin was polymerized in approximately 90 seconds, indicating the efficiency of the crosslinking solution of the glutaraldehyde was undisturbed.

Experiment No. 2/Uses of the Sealant

Pulmonary

A rabbit was used and an experimental model for the evaluation of the material as a pulmonary sealant.

A sealant, consisting of albumin, collagen, oleic acid, PEI and chitosan and crosslinked with heat-processed glutaraldehyde, was prepared in accordance with the method of invention. Concentrations for each ingredient were consistent with the values indicated in the above examples.

The lungs of an anaesthetized rabbit were exposed and deflated. Following, a portion of the upper lobe of the lung was transected and the cut site of the deflated lung was sealed and reinflated. The lung was evaluated for leakage by submersion in water. Evaluation of the lung for air leakage did not indicate any to be present, indicating the efficacy of the sealant.

Vascular

A rabbit was again used as an experimental model for the evaluation of the material as a vascular sealant.

In this experiment, the carotid arteries of an anesthetized, anticoagulated rabbit were bilaterally exposed. The artery of the left side was punctured with a 14 F catheter. Following removal of the catheter the hole was closed using the sealant. Alternately, the artery of the right side was transected, and a anastomosis was created using 6-0 Prolene suture. An umbilical tape was partially looped around the vessel proximal to the surgery site to momentarily reduce blood flow.

Sealant formulated to be consistent with the ranges heretofore indicated was applied to the puncture site using a tipped syringe. Following three minutes, the pressure was released to expose the repair to the full systolic/diastolic pressure of the carotid artery. No leakage was found to be present from the wound site.

Sealant formulated to be consistent with the ranges indicated was applied to the partially leaking anastomotic site of the right side of the experimental model. Following three minutes it was noted that the leakage stopped.

In a further experiment, a human cadaveric model was assessed for adhesion of the sealant onto the dura mater.

Following a craniotomy, the exposed dura was incised. Incision of the dura resulted in retraction of the tissue. The retracted tissue was drawn together, again using temporary stay sutures such that the incised edges were juxtaposed to one another. Sealant consistent with the formulations noted for this invention was prepared. The sealant was applied over the incision wound and the suture stays were released. The opposing edges of the incision wound remained aligned with one another, the sealant demonstrating adequate tenacity to resist the retractive forces of the dura. The cadavers head was lowered placing additional stress on the suture and the site was observed for failure of the sealant to hold the edges together. No failures were noted.

The present invention may be embodied in other specific forms without departing from the spirit or essential attributes thereof, and, accordingly, reference should be made to the appended claims, rather than to the foregoing specification, as an indication of the scope of the invention.

This invention has been described herein in considerable detail in order to comply with the patent statutes and to provide those skilled in the art with the information needed to apply the novel principles and to construct and use such specialized components as are required. However, it is to be understood that the invention can be carried out by specifically different equipment and devices, and that various modifications, both as to the equipment and operating procedures, can be accomplished without departing from the scope of the invention itself.

What is claimed is:

1. A soft tissue sealant formulation comprising:
(a) a substrate material comprising:
(i) an amount of a proteinaceous material,
(ii) an amount of a carbohydrate;
(iii) an adhesion modifier; and
(b) a crosslinking agent.

2. A sealant formulation as in claim 1 wherein said substrate further comprises an amount of a plasticizer.

3. A sealant formulation as in claim 1 wherein said proteinaceous material is selected from the group consisting of albumin, elastin and soluble and insoluble forms of collagen and combinations thereof.

4. A sealant formulation as in claim 1 wherein said carbohydrate material is selected from the group consisting of chitin, chitosan and derivatives of chitosan and combinations thereof.

5. A sealant material as in claim 3 wherein the protein concentration is between 1 and 50% (w/w) of said substrate material.

6. A sealant formulation as in claim 2 wherein the plasticizer is chosen from the group consisting of polyethylene glycol, glycerin, oleic acid, palmitic acid and combinations thereof.

7. A sealant formulation as in claim 6 wherein the plasticizer is preferably between 1 and 5% (w/w) of the substrate material.

8. A sealant formulation as in claim 1 wherein said adhesion modifier is selected from the group consisting of polyethyleneimine, gelatin and carboxymethylcellulose.

9. A sealant formulation as in claim 8 wherein said adhesion modifier is polyethyleneimine.

10. A sealant formulation as in claim 9 wherein the polyethyleneimine concentration is preferably between 1 and 4% (w/w) of said substrate material.

11. A sealant formulation as in claim 4 wherein said carbohydrate concentration is between about 1 and 5% (w/w) of said substrate material.

12. A sealant formulation as in claim 1 wherein said crosslinking agent comprises an aldehyde.

13. A sealant formulation as in claim 1 wherein said crosslinking agent comprises a dicarbonyl compound.

14. A sealant formulation as in claim 13 wherein said crosslinking agent is selected from the group consisting of succinaldehyde, glutaraldehyde and glutaric acid and mixtures thereof.

15. A sealant formulation as in claim 1 wherein said crosslinking agent comprises glutaraldehyde.

16. A sealant formulation as in claim 15 wherein said glutaraldehyde is heat stabilized.

17. A sealant formulation as in claim 2 wherein said crosslinking agent includes heat stabilized glutaraldehyde.

18. A sealant as in claim 1 further comprising an amount of buffer.

19. A sealant as in claim 18 further comprising an amount of buffer.

20. A sealant formulation as in claim 1 in kit form wherein said substrate comprising items (a)(i)-(a)(iii) is stored separate from said crosslinker for combination at the time of use.

21. A sealant formulation for use in wet or dry fields comprising:
(a) a subtrate including:
(i) an amount of proteinaceous material comprising a mixture of a major fraction of albumin and a minor fraction of collagen;
(ii) an amount of a carbohydrate procoagulant material including an amount of acetylated chitosan;
(iii) an adhesion modifying agent including polyethyleneimine;
(iv) an amount of a plasticizing agent selected from the group consisting of polyethylene glycol, glycerin, oleic acid, palmitic acid and combinations thereof; and
(b) an amout of a crosslinking agent including heat stabilized glutaraldehyde.

22. A sealant formulation as in claim 21 further comprising an amount of phosphate buffer.

23. A sealant formulation as in claim 21 in kit form wherein said substrate comprising items (a)(i)-(a)(iv) is stored separate from said crosslinker for combination at the time of use.

24. A sealant formulation as in claim 23 wherein said substrate further comprises an amount of a plasticizer material.

25. A sealant formulation as in claim 20 wherein said substrate and said crosslinker are provided in separate syringes.

26. A sealant formulation as in claim 23 wherein said substrate and said crosslinker are provided in separate syringes.

27. A sealant formulation as in claim 24 wherein said substrate and said crosslinker are provided in separate syringes.

28. A method of sealing tissue against gaseous or fluid leakage comprising steps of:
(a) admixing a sealant material including a substrate with a crosslinking agent, said substrate further including:
(i) an amount of a proteinaceous material,
(ii) an amount of a carbohydrate procoagulant;
(iii) an adhesion modifying agent;
(iv) a plasticizing agent; and
(b) applying said admixture to the surfaces of the tissue sought to be sealed.

29. A method as in claim 28 wherein said sealant is applied to a surgical field in which blood is present to control leakage.

30. A method as in claim 28 wherein said sealant is applied to tissue that is devoid of blood to control a subsequent fluid leakage.

31. A method of heat stabilizing glutaraldehyde, said method comprising steps of:
(a) preparing a stock solution of glutaraldehyde and water in which the concentration of the glutaraldehyde is approximately 1-20% (w/w);
(b) placing the solution into a closed vessel under a nitrogen head;
(c) heating the solution to a temperature of 35° C.-60° C. for a period of about 1-14 days; and
(d) allowing the heat-treated solution to cool to room temperature.

32. A method as in claim 31 wherein said stack solution is about 7-12% glutaraldehyde and at pH 7.01 and wherein the heating time is in the range of 72-120 hours.
